(19) United States
(12) Patent Application Publication
(10) Pub. No.: US 2020/0225187 A9
(43) Pub. Date: Jul. 16, 2020

(54) BIO-SENSING DEVICE

(71) Applicant: NDD, INC., Gyeongsangbuk-do (KR)

(72) Inventors: Sae Young AHN, Seoul (KR); Hyun Hwa KWON, Gyeongsangbuk-do (KR)

(21) Appl. No.: 16/099,809

(22) PCT Filed: Aug. 24, 2018
(86) PCT No.: PCT/KR2018/009814

§ 371 Date: Nov. 8, 2018

(30) Foreign Application Priority Data
Dec. 21, 2017 (KR) 10-2017-0177016

(51) Int. Cl. G01N 27/414; G01N 33/543

(52) U.S. Cl. CPC G01N 27/4145; H01L 51/0048; G01N 33/5438; G01N 27/4146

(57) ABSTRACT

The present invention provides a bio-sensing device comprising: a source electrode and a drain electrode disposed apart from each other; a gate electrode disposed apart between the source electrode and the drain electrode; an insulating film pattern disposed on the gate electrode to electrically insulate the gate electrode from the source electrode and the drain electrode; a sensing film, which is a channel connecting the source electrode and the drain electrode and is disposed on the insulating film pattern and on at least parts of the source electrode and the drain electrode and of which material includes single-walled carbon nanotubes (SWCNTs); and an anchor structure, which is a structure for binding the sensing film to the insulating film pattern and is disposed on the insulating film pattern and of which material includes multi-walled carbon nanotubes (MWCNTs).

### TECHNICAL FIELD

The present invention relates to a bio-sensing device, and more particularly, to a bio-sensing device having an electrode structure.

### BACKGROUND ART

Test methods used for the diagnosis of diseases are mainly based on coloration, fluorescence, etc. by enzyme reaction, but recently, immunoassay using an immune response between an antigen and an antibody has also been used. In the conventional immunoassay, optical measurement methods in which optical labelling is in combination with catalytic reaction of enzyme were used the most. These methods have disadvantages in that they require a complicated procedure that can be performed mainly by a laboratory-oriented and skilled researcher, the apparatus for analysis is large and expensive, and the analysis takes a long time.

### DISCLOSURE OF THE INVENTION

### Technical Problem

The present invention has been made to solve a lot of problems including the above ones, by providing a bio-sensing device that is capable of maximizing a performance of a sensing film and shortening analysis time and is relatively inexpensive. However, these problems are for illustrative purposes only, and the scope of the present invention is not limited thereto.

### Technical Solution

There is provided a bio-sensing device according to an aspect of the present invention in order to solve the above-described problems. The bio-sensing device comprises: a source electrode and a drain electrode disposed apart from each other; a gate electrode disposed apart between the source electrode and the drain electrode; an insulating film pattern disposed on the gate electrode so as to electrically insulate the gate electrode from the source electrode and the drain electrode; a sensing film, which is a channel connecting the source electrode and the drain electrode and is disposed on the insulating film pattern and on at least parts of the source electrode and the drain electrode and of which material includes single-walled carbon nanotubes (SWCNTs); and an anchor structure, which is a structure for binding the sensing film to the insulating film pattern and is disposed on the insulating film pattern and of which material includes multi-walled carbon nanotubes (MWCNTs).

There is provided a bio-sensing device according to another aspect of the present invention in order to solve the above-described problems. The bio-sensing device comprises: a source electrode and a drain electrode disposed apart from each other; a gate electrode disposed apart between the source electrode and the drain electrode; an insulating film pattern disposed on the gate electrode so as to electrically insulate the gate electrode from the source electrode and the drain electrode; a sensing film, which is a channel connecting the source electrode and the drain electrode and is disposed on the insulating film pattern and on at least parts of the source electrode and the drain electrode and of which material includes single-walled carbon nanotubes (SWCNTs); and an anchor structure, which is a structure for binding the sensing film to the insulating film pattern and is disposed on the insulating film pattern and of which material includes any one selected from graphene, gold (Au), and palladium (Pd).

In the bio-sensing device, the anchor structure includes a plurality of line patterns spaced apart from each other and the longitudinal direction of the line patterns may be perpendicular to the direction connecting the source electrode and the drain electrode.

In the bio-sensing device, the anchor structure includes a plurality of line patterns spaced apart from each other and the longitudinal direction of the line patterns may be parallel to the direction connecting the source electrode and the drain electrode.

In the bio-sensing device, the anchor structure may include a plurality of closed polygon-shaped patterns spaced apart from each other.

The bio-sensing device may further comprise a receptor that is attached to the sensing film and capable of binding to a target material.

In the bio-sensing device, the receptor may be attached to the sensing film by a functional group and may be any one or more selected from the group consisting of an enzyme substrate, a ligand, an amino acid, a peptide, an aptamer, a protein, a nucleic acid, a lipid and a carbohydrate.

In the bio-sensing device, the functional group may be at least one selected from the group consisting of an amine group, a carboxyl group and a thiol group.

In the bio-sensing device, the target material may be at least one selected from the group consisting of a protein, a peptide, an aptamer, a nucleic acid, an oligosaccharide, an amino acid, a carbohydrate, a dissolved gas, a sulfur oxide gas, a nitrogen oxide gas, a residual pesticide, a heavy metal and an environmentally harmful substance.

### Advantageous Effects

According to the embodiments of the present invention as described above, it is possible to provide a bio-sensing device that is capable of maximizing a performance of a sensing film and shortening analysis time and is relatively inexpensive. Of course, the scope of the present invention is not limited by these effects.

### BRIEF DESCRIPTION OF THE DRAWINGS

FIG. 1A and FIG. 1B are a sectional view and a plan view illustrating an aspect of an anchor structure in a process for preparing a bio-sensing device according to one embodiment of the present invention.

FIG. 1C and FIG. 1D are plan views illustrating an aspect of an anchor structure in a process for preparing a bio-sensing device according to other embodiments of the present invention.

FIG. 2A and FIG. 2B are a sectional view and a plan view illustrating the state in which a sensing film bound with an anchor structure is formed in a process for preparing a bio-sensing device according to one embodiment of the present invention.

FIG. 3 is a cross-sectional view illustrating a bio-sensing device according to an embodiment of the present invention.

### MODE FOR CARRYING OUT THE INVENTION

Hereinafter, embodiments of the present invention will be described in detail with reference to the accompanying drawings. Throughout the specification, it can be understood that when an element, such as a film, pattern, region, or substrate, is referred to as being placed “on” another element, the element may be directly “connected to” the other element or another intervening element may be present therebetween. In contrast, it is understood that when an element is referred to as being placed “directly on” another element, intervening elements are not present therebetween.

In the drawings, variations from the shapes of the illustrations as a result, for example, of manufacturing techniques and/or tolerances, can be expected. Thus, embodiments of the present invention should not be construed as limited to the particular shapes of regions illustrated herein but are to include deviations in shapes that result, for example, from manufacturing. Furthermore, the thickness or size of each layer in the drawings may be exaggerated for convenience and clarity of explanation. Like numerals refer to like elements.

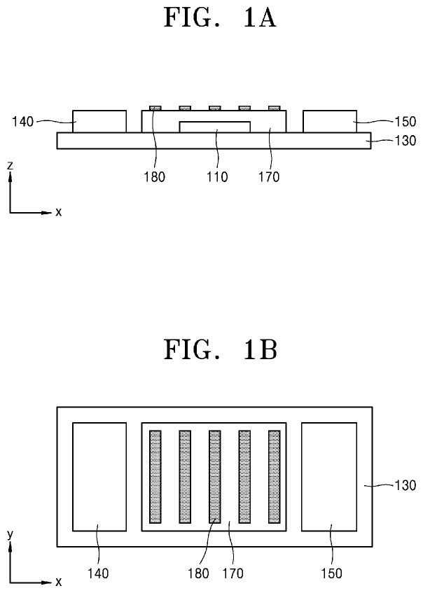

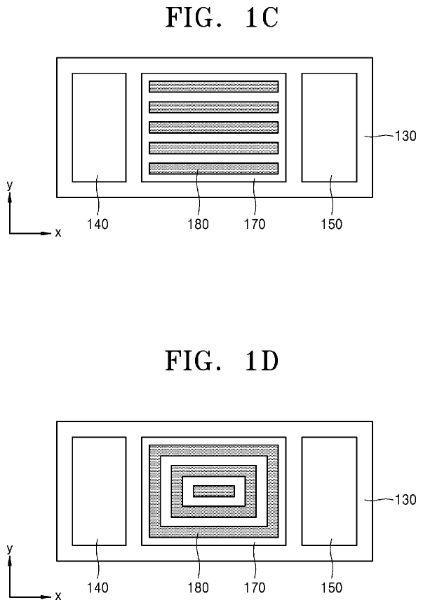

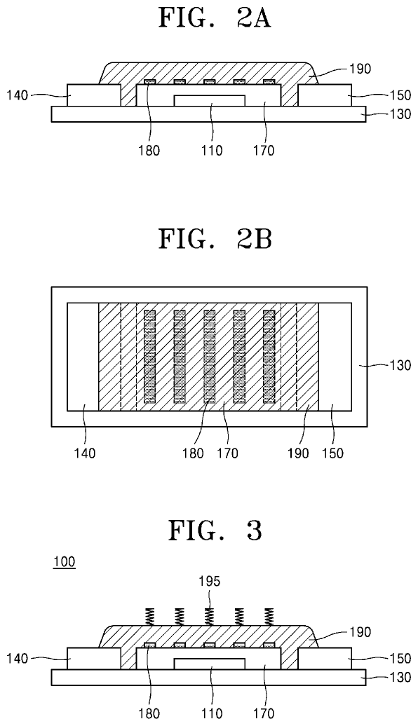

FIG. 1A and FIG. 1B are a sectional view and a plan view illustrating an aspect of an anchor structure in a process for preparing a bio-sensing device according to one embodiment of the present invention; FIG. 2A and FIG. 2B are a sectional view and a plan view illustrating the state in which a sensing film bound with an anchor structure is formed in a process for preparing a bio-sensing device according to one embodiment of the present invention; and FIG. 3 is a cross-sectional view illustrating a bio-sensing device according to an embodiment of the present invention.

Referring to FIGS. 1A and 1B, the bio-sending device according to one embodiment of the present invention comprises: a source electrode 140 and a drain electrode 150 disposed apart from each other on a substrate 130; a gate electrode 110 disposed apart between the source electrode 140 and the drain electrode 150; an insulating film pattern 170 disposed on the gate electrode 110 so as to electrically insulate the gate electrode 110 from the source electrode 140 and the drain electrode 150.

The source electrode 140, the drain electrode 150 and the gate electrode 110 may be made of an electrically conductive material, and for example, the material may include gold (Au). Although not shown in drawings, a barrier layer (for example, a Ti layer) may be interposed between the source electrode 140 and the substrate 130, and between the drain electrode 150 and the substrate 130. The insulating film pattern 170 may be an aluminum oxide (Al2O3) pattern, for example.

Furthermore, in a bio-sensing device according to one embodiment of the present invention, as a structure for binding a sensing film 190 to be formed in a subsequent process to the insulating film pattern 170, an anchor structure 180 is formed on the insulating film pattern 170.

Referring to FIG. 2A and FIG. 2B, in a bio-sensing device according to one embodiment of the present invention, as a channel connecting the source electrode 140 and the drain electrode, the sensing film 190 is formed on the insulating film pattern 170 and on at least parts of the source electrode 140 and the drain electrode 150.

The material of the sensing film 190 may include, for example, single-walled carbon nanotubes (SWCNTs). In this case, the sensing film 190 is implemented by supplying a precursor solution in a liquid state to a region including a space between the source electrode 140 and the drain electrode 150 and then solidifying the precursor solution. The solidification process may include at least one process selected from natural drying, heat drying and blow drying.

The anchor structure 180 is a structure for binding the sensing film 190 with the insulating film pattern 170 during the solidification of the precursor solution in a liquid state for forming the sensing film 190. In particular, when the sensing film 190 comprises single-walled carbon nanotubes (SWCNTs) and the anchor structure 180 comprises multi-walled carbon nanotubes (MWCNTs), the binding of single-walled carbon nanotubes (SWCNTs) and multi-walled carbon nanotubes (MWCNTs) becomes firmer and thus, the effect that the sensing film 190 is more strongly bound to the insulating film pattern 170 can be caused. It has been found that the binding power of single-walled carbon nanotubes (SWCNTs) and multi-walled carbon nanotubes (MWCNTs) is enhanced through a sort of homogeneous bonding, and the present invention uses this phenomenon.

Meanwhile, carbon nanotubes have a good charge transfer property and a large aspect ratio so as to secure a great number of charge transfer passages, thereby achieving high charge mobility and high transparency at the same time, and have good elasticity so as to be electrically and mechanically stable against large bending.

Single-walled carbon nanotubes (SWCNTs) have semiconductor characteristics and multi-walled carbon nanotubes (MWCNTs) have conductor characteristics, and thus, it is preferable that the sensing film 190 constituting the channel is formed with single-walled carbon nanotubes (SWCNTs) having semiconductor characteristics. However, it was confirmed that the anchor structure 180, which consists of multi-walled carbon nanotubes (MWCNTs) so as to have a conducting electric characteristic, plays an important role in interconnecting unitary strands constituting single-walled carbon nanotubes (SWCNTs) so as to secure a semiconducting electric path.

Meanwhile, as an alternative example, when the material of the sensing film 190 includes single-walled carbon nanotubes (SWCNTs), the material of the anchor structure 180 may also include any one selected from graphene, gold (Au), and palladium (Pd).

Meanwhile, the anchor structure 180 also serves to receive the precursor solution in a liquid state to be supplied for forming the sensing film 190. That is, the anchor structure 180 may serve to restrict the precursor solution to a desired predetermined region while preventing the precursor solution from flowing to an undesired region in the process of solidifying the precursor solution supplied in an ink jet mode into the region including the space between the source electrode 140 and the drain electrode 150.

The anchor structure 180 serving the above-described various functions may have various shapes on the insulating film pattern 170.

Referring to FIG. 1B, the anchor structure 180 includes a plurality of line patterns spaced apart from each other and the longitudinal direction (y direction) of the line patterns may be perpendicular to the direction connecting the source electrode and the drain electrode (x direction).

Referring to FIG. 1C, the anchor structure 180 includes a plurality of line patterns spaced apart from each other and the longitudinal direction (x direction) of the line patterns may be parallel to the direction connecting the source electrode and the drain electrode (x direction).

Referring to FIG. 1D, the anchor structure 180 may include a plurality of closed polygon-shaped patterns spaced apart from each other.

Meanwhile, although not shown in the drawings, in the source electrode 140 and the drain electrode 150, regions in contact with the sensing film 190 may have a comb-like shape. According to this structure, the bonding force or interconnectivity between the sensing film 190 and the electrodes 140, 150 can be improved.

Referring to FIG. 3, the receptor 195 may be attached to the sensing film 190 by a functional group. For example, the receptor 195 may be any one or more selected from the group consisting of an enzyme substrate, a ligand, an amino acid, a peptide, an aptamer, a protein, a nucleic acid, a lipid and a carbohydrate. Meanwhile, the functional group may be at least one selected from the group consisting of, for example, an amine group, a carboxyl group and a thiol group. The target material may be selected from the group consisting of, for example, a protein, an aptamer, a peptide, a nucleic acid, an oligosaccharide, an amino acid, a carbohydrate, a dissolved gas, a sulfur oxide gas, a nitrogen oxide gas, a residual pesticide, a heavy metal and an environmentally harmful substance.

The sensing film 190 may be made of a material that can vary in resistance depending on the receptor 195 and a target material bound to the receptor. The material of the sensing film 190 may include, for example, graphene, molybdenum disulfide (MoS2), or phosphorene, in addition to single-walled carbon nanotubes (SWCNTs). Meanwhile, in the bio-sensing device according to a modified embodiment of the present invention, the sensing film 190 may be made of a material that can vary in resistance by reacting directly with the target material without interposing the receptor 195.

The bio-sensing device according to an embodiment of the present invention can be used as an inspection device that is used for disease diagnosis and can be used as a sensing device that uses an immune reaction between an antigen and an antibody depending on the kind of a sensing film and a receptor. In this case, it is advantageous that, since the result of electrical measurement is utilized, a complicated procedure is not required in the analysis process, the apparatus for analysis is relatively inexpensive, and the analysis does not take a long time.

While the present invention has been particularly shown and described with reference to embodiments shown in the drawings, it is only for illustrative purposes. It will be understood by those skilled in the art that various modifications and equivalent embodiments may be made. Therefore, the scope of the present invention should be determined by the technical idea of the appended claims.

1. A bio-sensing device comprising:
a source electrode and a drain electrode disposed apart from each other;
a gate electrode disposed between the source electrode and the drain electrode;
an insulating film pattern disposed on the gate electrode to electrically insulate the gate electrode from the source electrode and the drain electrode;
a sensing film, the sensing film being a channel connecting the source electrode and the drain electrode and being disposed on the insulating film pattern and on at least parts of the source electrode and the drain electrode, of which material includes single-walled carbon nanotubes (SWCNTs); and
an anchor structure, the anchor structure being a structure for binding the sensing film to the insulating film pattern and being disposed on the insulating film pattern, of which material includes multi-walled carbon nanotubes (MWCNTs).

2. A bio-sensing device comprising:
a source electrode and a drain electrode spaced apart from each other;
a gate electrode disposed between the source electrode and the drain electrode;
an insulating film pattern disposed on the gate electrode to electrically insulate the gate electrode from the source electrode and the drain electrode;
a sensing film, the sensing film being a channel connecting the source electrode and the drain electrode and being disposed on the insulating film pattern and on at least parts of the source electrode and the drain electrode, of which material includes single-walled carbon nanotubes (SWCNTs); and
an anchor structure, the anchor structure being a structure for binding the sensing film to the insulating film pattern and being disposed on the insulating film pattern, of which material includes any one selected from graphene, gold (Au), and palladium (Pd).

3. The bio-sensing device of claim 1, wherein the anchor structure includes a plurality of line patterns spaced apart from each other and the longitudinal direction of the line patterns is perpendicular to the direction connecting the source electrode and the drain electrode.

4. The bio-sensing device of claim 1, wherein the anchor structure includes a plurality of line patterns spaced apart from each other and the longitudinal direction of the line patterns is parallel to the direction connecting the source electrode and the drain electrode.

5. The bio-sensing device of claim 1, wherein the anchor structure includes a plurality of closed polygon-shaped patterns spaced apart from each other.

6. The bio-sensing device of claim 1, further comprising:
a receptor that is attached to the sensing film and capable of binding to a target material.

7. The bio-sensing device of claim 6, wherein the receptor is attached to the sensing film by a functional group and is any one or more selected from the group consisting of an enzyme substrate, a ligand, an amino acid, a peptide, an aptamer, a protein, a nucleic acid, a lipid and a carbohydrate.

8. The bio-sensing device of claim 7, wherein the functional group is at least one selected from the group consisting of an amine group, a carboxyl group and a thiol group.

9. The bio-sensing device of claim 6, wherein the target material is at least one selected from the group consisting of a protein, a peptide, an aptamer, a nucleic acid, an oligosaccharide, an amino acid, a carbohydrate, a dissolved gas, a sulfur oxide gas, a nitrogen oxide gas, a residual pesticide, a heavy metal and an environmentally harmful substance.

10. The bio-sensing device of claim 2, wherein the anchor structure includes a plurality of line patterns spaced apart from each other and the longitudinal direction of the line patterns is perpendicular to the direction connecting the source electrode and the drain electrode.

11. The bio-sensing device of claim 2, wherein the anchor structure includes a plurality of line patterns spaced apart from each other and the longitudinal direction of the line patterns is parallel to the direction connecting the source electrode and the drain electrode.

12. The bio-sensing device of claim 2, wherein the anchor structure includes a plurality of closed polygon-shaped patterns spaced apart from each other.

13. The bio-sensing device of claim 2, further comprising:
a receptor that is attached to the sensing film and capable of binding to a target material.

14. The bio-sensing device of claim 13, wherein the receptor is attached to the sensing film by a functional group and is any one or more selected from the group consisting of an enzyme substrate, a ligand, an amino acid, a peptide, an aptamer, a protein, a nucleic acid, a lipid and a carbohydrate.

15. The bio-sensing device of claim 14, wherein the functional group is at least one selected from the group consisting of an amine group, a carboxyl group and a thiol group.

16. The bio-sensing device of claim 13, wherein the target material is at least one selected from the group consisting of a protein, a peptide, an aptamer, a nucleic acid, an oligosaccharide, an amino acid, a carbohydrate, a dissolved gas, a sulfur oxide gas, a nitrogen oxide gas, a residual pesticide, a heavy metal and an environmentally harmful substance.
